(19) United States
(12) Patent Application Publication
(10) Pub. No.: US 2009/0105675 A1
(43) Pub. Date: Apr. 23, 2009

(54) Portable Urine Collection Device

(76) Inventor: Bradley Rex Romero, Auburn, CA (US)

Correspondence Address: SUPERSLAB 100 CORP, 745 SOUTH HIGHWAY 65, SUITE 30, LINCOLN, CA, 95648, US

(21) Appl. No.: 11/309,939

(22) Filed: Nov. 9, 2006

(51) Int. Cl. A61F 5/453

(52) U.S. Cl. 604/349

(57) ABSTRACT

A portable urine collection device for males that will allow a male in a vehicle to relieve himself discreetly and sanitarily without having to leave the passenger compartment of the vehicle. The portable urine collection device for males includes a mainframe container having a top and bottom cylindrical walls and also having a cap being removable and likewise replaceable. Located within the device is an organic, biodegradable, granular absorbent component. Said component being capable of liquid absorption at a rate six times its own weight. A portable urine collection device that is utilized in a vehicle and becomes completely biodegradable when discarded.

### BACKGROUND OF THE INVENTION

1. Field of the Invention

The present invention relates to a male urine collection device and more particularly pertains to a new portable urine collection device for males allowing a male passenger in a vehicle to relieve himself discreetly and sanitarily without having to exit the vehicle. Said device is then stored and discarded appropriately at a later time. Furthermore, in the event the portable urine collection device was disposed of improperly its components and nature is completely biodegradable.

2. Description of the Prior Art

The use of a male urine collection system is known in the prior art. More specifically, male urine collection systems heretofore devised and utilized are known to consist basically of familiar, expected and obvious structural configurations, notwithstanding the myriad of designs encompassed by the crowded prior art which have been developed for the fulfillment of countless objectives and requirements. Known prior art includes U.S. Pat. No. 4,713,067; U.S. Pat. No. 4,073,295; U.S. Pat. No. 5,312,383; U.S. Pat. No. 5,318,550; U.S. Pat. No. 5,439,456; U.S. Pat. No. 6,007,521; and U.S. Pat. No. Des. 357,979.

While these devices fulfill their respective, particular objectives and requirements, the aforementioned patents do not disclose a new portable urine collection device for males. The prior art includes funnels, hoses, bladders and collection devices for collecting urine.

### SUMMARY OF THE INVENTION

The general purpose of the present invention, which will be described subsequently in greater detail, is to provide a new portable urine collection device for males which has many of the advantages of the male urine collection systems mentioned heretofore and many novel features that result in a new portable urine collection device for males which is not anticipated, rendered obvious, suggested, or even implied by any of the prior art male urine collection systems, either alone or in any combination thereof. The present invention includes a biodegradable, cylindrical container mainframe having a bottom rolled inner edge utilizing a seal (disc) and also having a cap (rolled inner edge & disc) being telescopic and removable and likewise resealable; Located within the device is an organic, biodegradable, granular component. Said component is capable of liquid absorption at a rate six times its weight. None of the prior art includes the combination of the elements of the present invention.

There has thus been outlined, rather broadly, the more important features of the portable urine collection device for males in order that the detailed description thereof that follows may be better understood, and in order that the present contribution to the art may be better appreciated. There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

It is an object of the present invention to provide a new portable urine collection device for males which has many of the advantages of the male urine collection systems mentioned heretofore and many novel features that result in a new portable urine collection device for males which is not anticipated, rendered obvious, suggested, or even implied by any of the prior art male urine collection systems, either alone or in any combination thereof.

Still another object of the present invention is to provide a new portable urine collection device for males for allowing a male passenger in a vehicle to relieve himself discreetly and sanitarily without having to leave the vehicle.

Still yet another object of the present invention is to provide a new portable urine collection device for males that is easy and convenient to use and completely biodegradable.

Even still another specific object of the present invention is to provide a new portable urine collection device for males that prevents urinary and bladder problems by the user having to avoid going to the bathroom until a suitable location is found while traveling and more factually significant, if the portable urine collection device is discarded improperly, it is known to be a completely biodegradable device and will have already converted the liquid into a solid state; said device will completely dissipate over a short period of time if discarded in this manner.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a perspective view of the mainframe assembly, telescopic cap assembly and the organic, biodegradable, absorbent of said portable urine collection device according to the present invention.

FIG. 2 is a perspective view of said portable urine collection device mainframe assembly of the present invention.

FIG. 3 is a perspective view of the telescopic cap assembly for said portable urine collection device of the present invention.

### DESCRIPTION OF THE PREFERRED EMBODIMENT

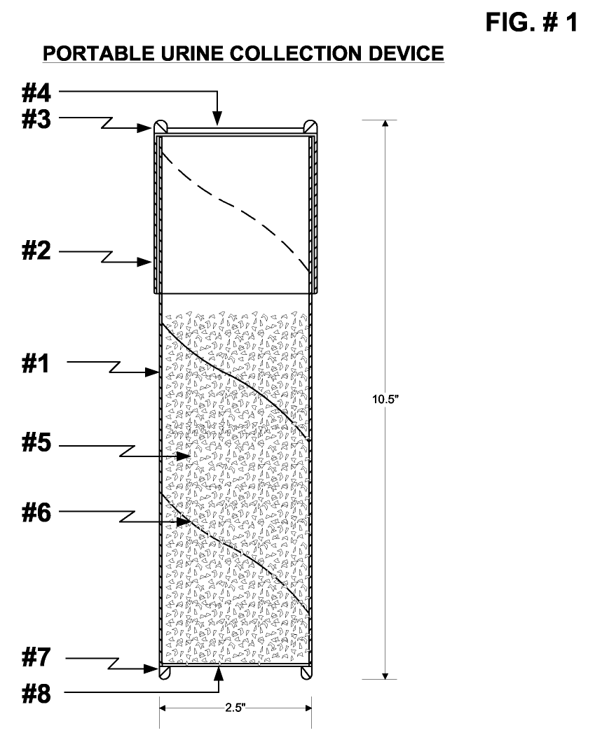

With reference now to the drawings, and in particular to FIGS. 1 through 3 thereof, a new portable urine collection device for males embodying the principles and concepts of the present invention will be described.

As best illustrated in FIG. 1, the inclusive portable urine collection device for males generally comprises a container device with a mainframe assembly #1 having an inward rolled bottom edge #7; said edge being sealed with a paper/cardboard disc #8 located within the walls of the cylinder; said disc #8 being seated firmly against the rolled inner edge #7 to accomplish closure of the bottom of said mainframe assembly. Construction of said mainframe container assembly #1 is accomplished by a glued seam #6. Said cap assembly #2 engaged for closure of mainframe assembly #1 with said organic, biodegradable absorbent contents #5 installed. Said organic, biodegradable, granular, compound #5 being located within the sealed device and capable of remarkable liquid absorption when ultimately deployed. Said cap #2 being removably and securely replaceable over the top opening of the mainframe assembly #1 after complete bladder void.

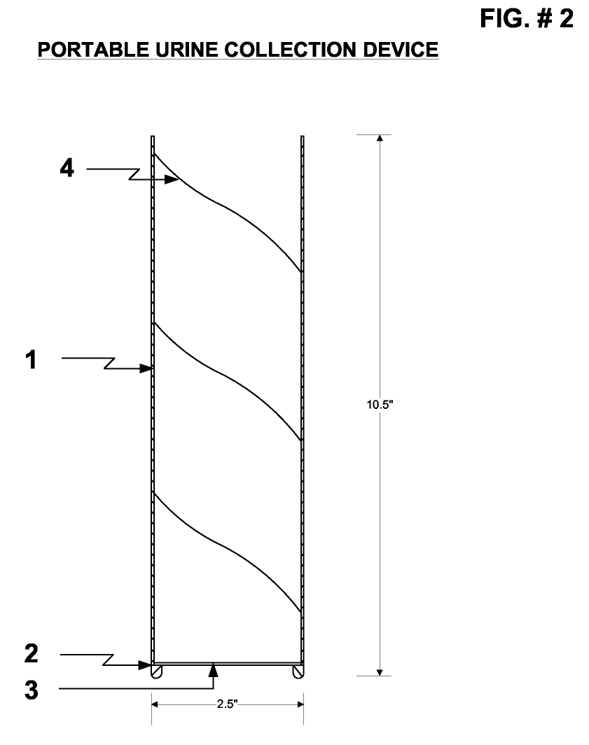

FIG. 2 the inclusive portable urine collection device for males generally comprises a container device including a container mainframe assembly #1 having an inward rolled bottom edge #2; said edge being sealed with a cardboard disc #3 located within the walls of the cylinder; said disc #3 being seated firmly against the rolled inner edge #2 to accomplish closure of the bottom of said mainframe assembly. Construction of said mainframe container assembly #1 is accomplished by a glued seam #4. Said cap assembly and organic, biodegradable, absorbent removed.

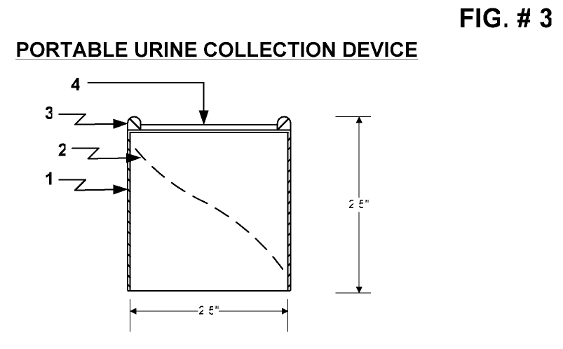

FIG. 3 Said telescopic cap #1 also being constructed identically as mainframe cylinder utilizing a glued seam #2; withstanding said cap #1 having a slightly larger inside diameter and shorter length, thus allowing a secure and sealed telescopic fit when applied to opened end of mainframe container assembly. Said cap likewise has a constructed rolled inner edge #3 and sealing disc #4.

In use, (Referencing FIG. 1) the user removes said cap #2 and placed one's male urinary organ into the opened end of the mainframe of said cylindrical portable urine collection device #1 and eliminates one's bladder into the container; whereupon the user reengages cap #2 onto mainframe assembly #1. The granular, organic, biodegradable absorbent #5 now solidifies from the introduction of urine. The user replaces the removable telescopic cap #2 engaging it onto the container #1. The portable urine collection device is then disposed of as a complete unit when appropriate. Furthermore and significantly important, the portable urine collection device is biodegradable by nature and will dissipate completely if discarded inappropriately.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shapes, form, function and manner of operation, device and use, are deemed readily apparent and obvious to one skilled in the art, and all equivalent relationships to those illustrated in the photographs and described in the specification are intended to be encompassed by the present invention.

Therefore, the foregoing is considered as illustrative only of the principles of the portable urine collection device for males. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A portable urine collection device for males comprising: a mainframe cylindrical container including a device having a stationary bottom disc and also including a telescopic cap being removable. This mainframe cylindrical device is constructed of thick paper/cardboard and further rolled and glued at its seams. Said seams rotate the length, being similar in nature to a barber pole rotation. The bottom edge of the cylinder is a rolled edge which becomes an inner edge around the circumference of the cylindrical bottom edge for said cardboard disc to seat upon. This disc is placed within the mainframe of the cylinder and becomes the bottom of the cylinder, closing the opened bottom end. The opposite end, or said top of the cylindrical container, is left opened void of a rolled edge and being capped by said telescopic cap manufactured of the same construction with a slightly larger inner diameter and being removably engaged. Further including an organic, biodegradable, granular component capable of liquid absorption at a rate six times its weight.

2. The portable urine collection device for males as described in claim #1, wherein said cylindrical paper tube includes a tubular-shaped container having an open end and a closed end, and also includes a tubular telescopic cap that when engaged seals the device and its organic contents.
